(19) United States
(12) Patent Application Publication
(10) Pub. No.: US 2026/0232185 A1
(43) Pub. Date: Aug. 13, 2026

(54) TEAR FILM STABILITY ASSESSMENT WITH INTEGRATED GRAPHICAL REPRESENTATION

(71) Applicant: Alcon Inc., Fribourg (CH)

(72) Inventors: Martin Gründig, Rangsdorf (DE); Corinna Both, Potsdam (DE); Armin Haupt, Berlin (DE); John Alfred Campin, St Cloud, FL (US); George Hunter Pettit, Fort Worth, TX (US); Kevin Baker, Duluth, GA (US)

(21) Appl. No.: 19/536,318

(22) Filed: Feb. 11, 2026

Related U.S. Application Data
(60) Provisional application No. 63/757,398, filed on Feb. 12, 2025.

(51) Int. Cl. A61B 3/12; A61B 3/00; A61B 3/14

(52) U.S. Cl. CPC A61B 3/12; A61B 3/0025; A61B 3/0041; A61B 3/14

(57) ABSTRACT

A method of assessing tear film stability in an ocular surface includes obtaining tear film breakup time data and blink time series for the ocular surface, via one or more imaging assemblies. The method includes converting the tear film breakup time data to a tear film breakup map represented as a polar data array, via a controller, such that respective elements in the polar data array represent a local tear film breakup time at respective radial and angular locations. The blink time series is converted to an interblink interval map such that respective elements in the interblink interval map represent an interblink time interval at different blink completeness levels. The tear film breakup map is integrated with the interblink interval map to generate a tear film stability map. The method includes directing a remedial action when an enabling condition related to the tear film stability map is met.

### INTRODUCTION

The disclosure relates generally to a method and system for assessing the tear film stability of an ocular surface using an integrated graphical representation. The ocular tear film is a thin layer of fluid that covers the surface of the eye, including the cornea, conjunctiva, and inner eyelids. The tear film is made up of three layers: an outer lipid layer that reduces evaporation, an aqueous middle layer that lubricates the eye and washes away particles, and an inner mucus layer that nourishes the cornea. If the tear film is not intact and stable, it may lead to less precise visual outcomes after a surgical procedure. For example, when patients receive advanced intraocular lens (IOL) implants during eye surgery, an unstable tear film may negatively impact the accuracy of their final vision correction. Complex screening approaches are generally required for assessments related to tear film stability.

### SUMMARY

Disclosed herein is a method of assessing tear film stability in an ocular surface with one or more imaging assemblies and a controller having a processor and tangible, non-transitory memory on which instructions are recorded. The method includes obtaining tear film breakup time data and blink time series for the ocular surface via the imaging assemblies. The method includes converting the tear film breakup time data to a tear film breakup map represented as a polar data array, via a controller, such that respective elements in the polar data array represent a local tear film breakup time at respective radial and angular locations. The blink time series is converted to an interblink interval map such that respective elements in the interblink interval map represent an interblink time interval at respective blink completeness levels. The method includes integrating the tear film breakup map with the interblink interval map to generate a tear film stability map and directing a remedial action when at least one enabling condition related to the tear film stability map is met.

Prior to integrating the tear film breakup map with the interblink interval map, the method may include matching a respective scale of the interblink interval map and the tear film breakup map, the interblink interval map and the tear film breakup map having a respective corneal apex position, via the controller. The interblink interval map and the tear film breakup map may be aligned based on the respective corneal apex position, via the controller. The method may include calculating the stability index as a ratio of the local tear film breakup time over the interblink time interval, via the controller. The stability index may be calculated as a difference between the local tear film breakup time and the interblink time interval, via the controller.

In some embodiments, the method includes setting the at least one enabling condition to be met when the stability index of at least one of the respective pixels inside a pupillary boundary in the tear film stability map is below a predefined pupil threshold. In other embodiments, the method includes identifying the respective pixels having a lowest value of the stability index as an instability maximum, via the controller, and setting the at least one enabling condition to be met when the instability maximum is inside a pupillary boundary.

In some embodiments, the method includes calculating a total area of the respective pixels in the tear film stability map having the stability index below a predefined area threshold, calculating a fraction of the total area to a total mapped ocular surface, and setting the at least one enabling condition to be met when the fraction is greater than a predefined maximum.

The method may include obtaining a global stability value as a weighted sum of the stability index for respective pixels in the tear film stability map such that weighting factors in the weighted sum are relatively higher in a central region and relatively lower in a peripheral region of the ocular surface, via the controller. The method may include setting the at least one enabling condition to be met when the global stability value is less than a predefined value. The method may include selecting the remedial action to include a therapeutic treatment for dry eyes and/or a screening for advanced intraocular lens implantation. The imaging assemblies may be selected to include a camera unit having a capturing frequency of at least 70 Hertz.

Disclosed herein is a system for assessing tear film stability of an ocular surface. The system includes one or more imaging assemblies adapted to respectively obtain tear film breakup time data and a blink time series for the ocular surface. The system includes a controller having a processor and tangible, non-transitory memory on which instructions are recorded. The controller is adapted to convert the tear film breakup time data to a tear film breakup map represented as a polar data array such that respective elements in the polar data array represents a local tear film breakup time at a respective radial and angular location. The controller is adapted to convert the blink time series to an interblink interval map such that the respective elements in the interblink interval map represent an interblink time interval at different blink completeness levels. The controller is adapted to integrate the tear film breakup map with the interblink interval map to generate a tear film stability map, including calculating a stability index for respective pixels in the tear film stability map. A remedial action is directed when at least one enabling condition related to the tear film stability map is met.

The above features and advantages and other features and advantages of the present disclosure are readily apparent from the following detailed description of the best modes for carrying out the disclosure when taken in connection with the accompanying drawings.

### BRIEF DESCRIPTION OF THE DRAWINGS

FIG. 1 is a schematic illustration of a system for assessing tear film stability of an ocular surface, the system having a controller;

FIG. 2 is a schematic diagram of an eye;

FIG. 3 is a schematic flowchart for a method executable by the controller of FIG. 1;

FIG. 4 is a schematic diagram of tear film breakup time data, displayed as a polar data array;

FIG. 5 is a schematic diagram illustrating an interblink interval map generated from a blink completeness time series; and

FIG. 6 is a schematic diagram of a tear film stability map generated by the controller of FIG. 1.

Representative embodiments of this disclosure are shown by way of non-limiting example in the drawings and are described in additional detail below. It should be understood, however, that the novel aspects of this disclosure are not limited to the particular forms illustrated in the above-enumerated drawings. Rather, the disclosure is to cover modifications, equivalents, combinations, sub-combinations, permutations, groupings, and alternatives falling within the scope of this disclosure as encompassed, for instance, by the appended claims.

### DETAILED DESCRIPTION

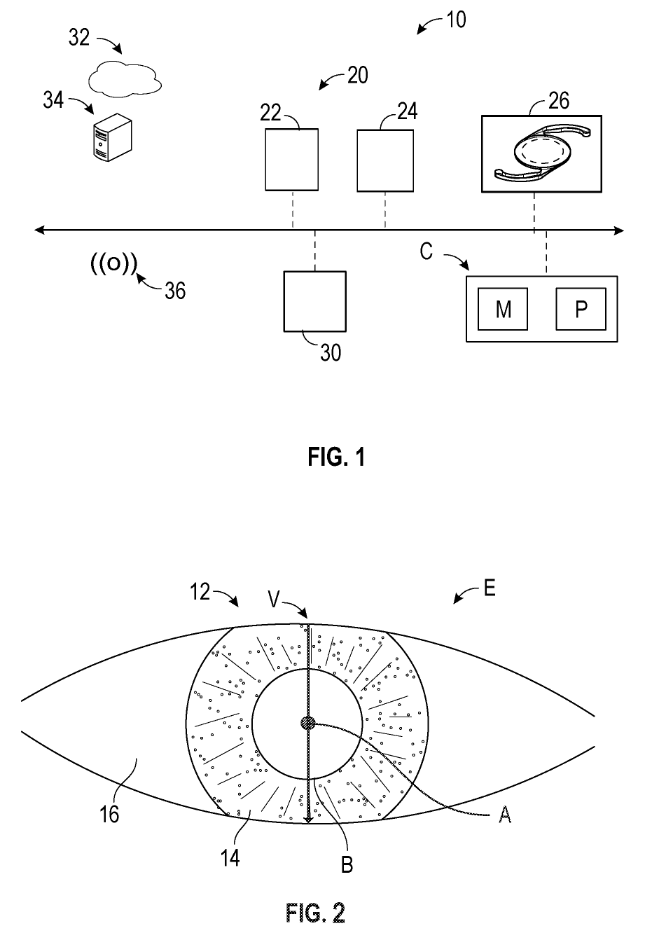

Referring to the drawings, wherein like reference numbers refer to like components, FIG. 1 schematically illustrates a system 10 for assessing tear film stability of an ocular surface in a subject. An example ocular surface 12 in an eye E (having cornea 14 and sclera 16) is shown in FIG. 2. As shown in FIG. 2, the ocular surface 12 defines a corneal apex A and covers a pupil region that is within a pupillary boundary B.

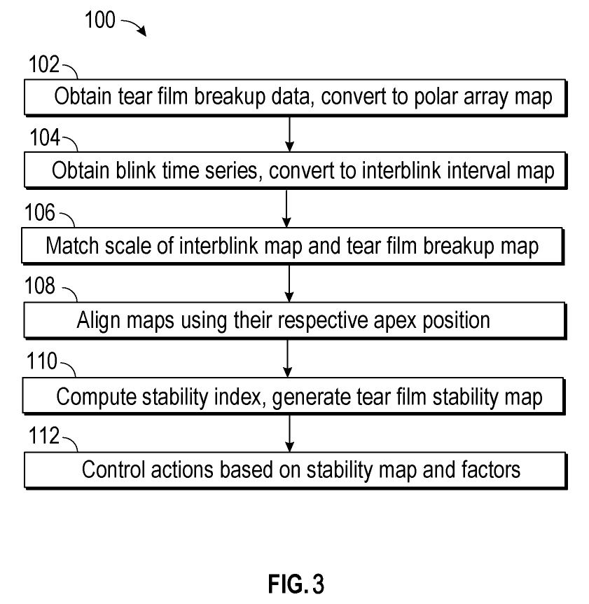

Referring to FIG. 1, the system 10 includes a controller C having at least one processor P and at least one memory M (or non-transitory, tangible computer readable storage medium) on which are recorded instructions for executing a method 100 of assessing tear film stability, which is shown in and described below with reference to FIG. 3. The memory M can store controller-executable instruction sets, and the processor P can execute the controller-executable instruction sets stored in the memory M.

Referring to FIG. 1, the system 10 includes one or more imaging assemblies 20 adapted to obtain tear film breakup time data and a blink time series for the ocular surface. For example, a first imaging assembly 22 is adapted to obtain non-invasive tear film breakup time data. Referring to FIG. 1, a second imaging assembly 24 adapted to obtain blink time series data. In some embodiments, the second imaging assembly 24 includes a camera system that can support quantitative blink analysis at a relatively high speed that is above a threshold speed. In one example, the threshold speed is 70 Hz. In another example, the threshold speed is 90 Hz. It is understood that the imaging assemblies 22 and 24 may be the same, with the non-invasive tear film breakup time data and blink time series data being recorded simultaneously or one after the other.

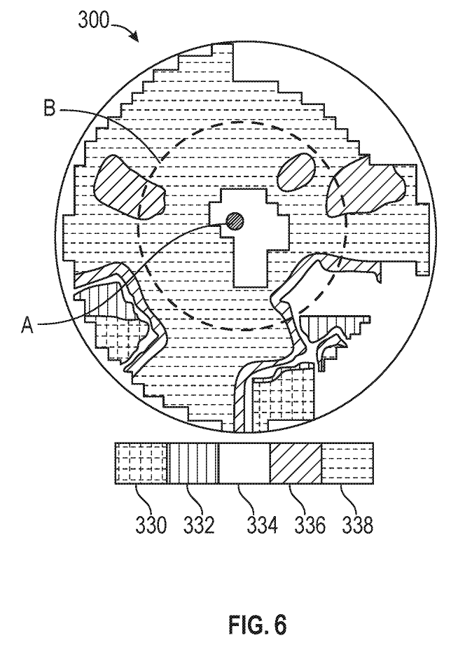

As described below, the system 10 provides a combined graphical representation (shown as tear film stability map 300 in FIG. 6) to simplify the assessment of tear film stability by integrating spatial-temporal data of independent tear film screening methods. The integrated graphical display integrates measurements representing complementary information on tear film regeneration/replenishment (e.g. blinking behavior) as well as tear film degradation (e.g. non-invasive tear film breakup time). The system 10 may be employed for screening of tear film stability assessment, dry eye disease screening, and as a screening tool to identify patients for whom advanced intraocular lens transplantation is indicated.

Referring now to FIG. 3, an example flowchart of the method 100 is shown, which may be dynamically executed and need not be applied in the specific order recited herein. It is understood that the steps may be concurrently executed. Method 100 may be embodied as computer-readable code or instructions stored on and partially executable by the controller C of FIG. 1. Furthermore, it is to be understood that some steps may be eliminated.

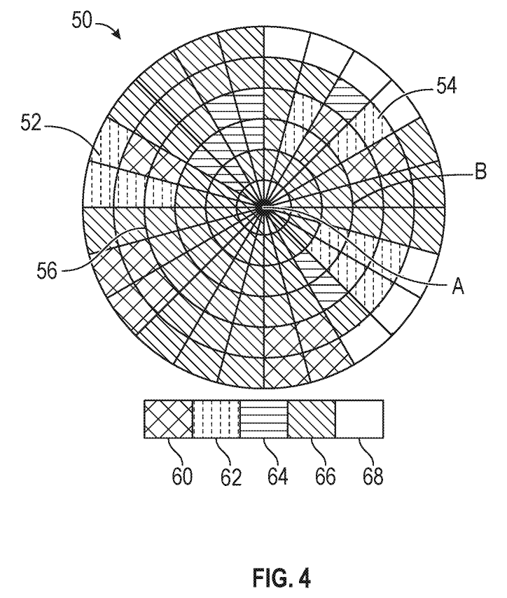

Per block 102 of FIG. 3, the method 100 includes obtaining tear film breakup time data and converting the tear film breakup time data to a tear film breakup map. FIG. 4 illustrates an example tear film breakup map 50. The tear film breakup time is defined as the time in seconds from a blink until the first appearance of tear film breakup. The subject is asked to suppress blinking during that time. The tear film breakup time is recorded as the number of seconds that elapse between the last blink and the appearance of the first dry spot in the tear film. The test may be performed invasively (e.g., through instillation of sodium fluorescein) or non-invasively (e.g., through placido-ring pattern reflection).

In the embodiment shown, the test is performed non-invasively as the instillation of fluorescein dye can itself alter tear film dynamics, potentially artificially modifying the test results. The non-invasive tear film breakup time test uses corneal topography instruments and video-keratography, capturing real-time changes in the tear film surface without the need for dye and direct eye contact. For example, the first imaging assembly 22 may include a specialized device projecting a grid or pattern onto the tear film, a camera that tracks how quickly this pattern becomes distorted, indicating tear film break-up.

Referring to FIG. 4, the tear film breakup map 50 is represented as a polar data array. The central spot is the corneal apex A. The array shape encodes spatial data as defined by radial and angular increments provided along with the array. In other words, the tear film breakup map 50 uses a polar data array to capture spatial variations in tear film stability, with each respective element (e.g., element 52, 54, 56) representing a local tear film breakup time at specific radial and angular locations. The patterns shown in the tear film breakup map 50 (shown in legend elements 60, 62, 64, 66, 68) correspond to different values of the local tear film breakup time, which may range from zero seconds to about 15 seconds. Other non-radial data representations may be employed.

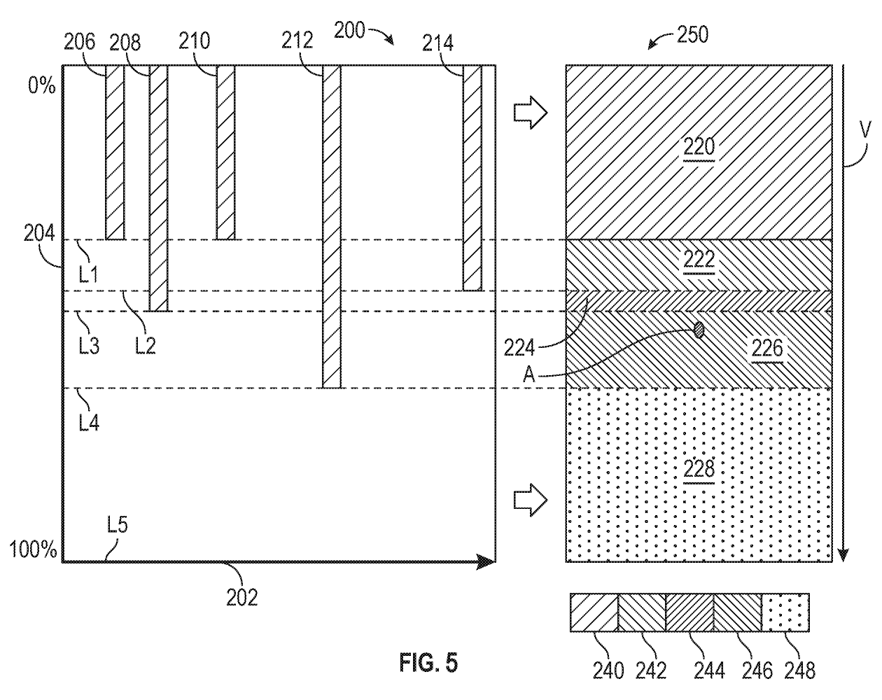

Per block 104 of FIG. 3, the method 100 includes obtaining a blink completeness time series, and converting the blink time series to an interblink interval map. FIG. 5 illustrates examples of a blink completeness time series 200 and an interblink interval map 250. The horizontal axis 202 in the blink completeness time series 200 indicates time in seconds, while the vertical axis 204 indicates blink completeness, ranging from 0 to 100 percent. The blink completeness time series 200 plots the quantitative reading of eye opening over the course of a period of time, e.g. 20 seconds. The bars 206, 208, 210, 212, 214 indicate individual blink events over time, at varying levels of completeness that is indicated by the horizontal lines L1, L2, L3, L4, L5 (from the lowest value to the highest value, respectively).

Referring to FIG. 5, the interblink interval map 250 shows respective interblink time interval at the blink completeness levels indicated by lines L1, L2, L3, L4, L5 so that each area of the ocular surface 12 will have a different value depending on the frequency of the blink. The time elapsed between blinks is the interblink interval. For each blink sequence, a meaninterblink interval (IBI)is calculated for each horizontal line.

In this example, the spatial distribution of the blink behavior is mapped along a vertical axis V (see FIG. 2 and 5), since blink completeness is computed only from one position of the eyelid. In other words, for each value from 0 to 100% of the blink completeness the mean interblink interval (measurement duration divided by the number of blinks at percent value) is calculated and transformed into a map. Referring to FIG. 5, the five zones 220, 222, 224, 226, 228 in the interblink interval map 250 correspond respectively to legend elements 240, 242, 244, 246, 248, each defining a different value of the interblink interval. By way of illustration only, assuming the total time period was 20 seconds, there are 5 blink events (bars 206, 208, 210, 212, 214) that have a completeness level that is at least at level L1, thus the interblink interval for zone 220 is calculated as 4 seconds (20/5). Similarly, there are 3 blink events (bars 208, 212, 214) that have a completeness level that is at least at level L2, thus the interblink interval for zone 222 is calculated as 6.7 seconds (20/3). Similarly, there are 2 blink events (bars 208, 212) that have a completeness level that is at least at level L3, thus the interblink interval for zone 224 is calculated as 10 seconds (20/2).

This method reveals subtle variations in blink patterns, distinguishing between complete lid closures and partial eye movements. Regions in the lower area of the cornea have a correspondingly higher interblink interval than regions located in the upper area if there are partial eye closures among the blinks. This requires also the absolute pixel values of the blink range in the image and the corneal apex A. The relative positions (percent completeness) are then transformed into pixel coordinates based on the provided upper and lower blink range pixel values.

Proceeding to block 106 of FIG. 3, the method 100 includes matching the respective scales of the tear film breakup map 50 (see FIG. 4) and the interblink interval map 250 (see FIG. 5). In other words, the spatial representations in the tear film breakup map 50 and the interblink interval map 250 are matched to the same scale. Advancing to block 108 of FIG. 3, the method 100 includes aligning tear film breakup map 50 and the interblink interval map 250 based their respective corneal apex position A. This alignment may be accomplished using other reference points, for example, the pupil center or the corneal vertex.

Proceeding to block 110 of FIG. 3, the method 100 includes integrating the tear film breakup map 50 with the interblink interval map 250 to generate a tear film stability map. FIG. 6 is a schematic diagram of a tear film stability map 300, annotated with the corneal apex A and the pupillary boundary B. In regions for which data from both measurements exist the local tear film stability, referred to herein as stability index, is computed. Accordingly, the size of the tear film stability map 300 resembles the shape of the smaller of the two data sources. It is understood that the FIGS. shown herein are not drawn to scale and are intended as examples only.

Each pixel in the tear film stability map 300 is defined by a stability index calculated by the controller C. In some embodiments, the stability index for each pixel is calculated as a ratio (TBUT/IBI) of the local tear film breakup time (TBUT) over the respective interblink time interval (IBI). In other embodiments, the stability index is calculated as a difference (TBUT- IBI) between the local tear film breakup time (TBUT) and the respective interblink time interval (IBI). Specific subregions of the tear film stability map 300 may be employed to determine additional metrics related to regions of interest.

Referring to FIG. 6, the various patterns shown correspond to different values or range of values of the stability index, as depicted in the legend elements 330, 332, 334, 336, 338. By way of example only, where the stability index for each pixel is calculated as a ratio (TBUT/IBI), the legend elements 330, 332, 334, 336, 338 may respectively represent a ratio of 1.2, 1.0, zero or no data available, 0.9, and 0.8.

Proceeding to block 112 of FIG. 3, the method 100 includes determining if at least one enabling condition related to the tear film stability map 300 is met and directing a remedial action when at least one enabling condition is met. The remedial action may include a therapeutic treatment for dry eye disease. The remedial action may include screening for advanced intraocular lens implantation, as dry eyes are a comorbidity for advanced intraocular lens implantation. In other words, the system 10 may be employed to identify patients for whom specific types of advanced intraocular lenses are indicated. For example, the tear film stability map 300 for each subject may be fed as an input factor to an intraocular lens selection module 26 that recommends an intraocular lens for the subject.

In one embodiment, the enabling condition is met when the stability index of at least one of the respective pixels inside a pupillary boundary B in the tear film stability map 300 is below a predefined pupil threshold. In another embodiment, the controller C may be adapted to identify the respective pixels having a lowest value of the stability index as an instability maximum, with the enabling condition being met when the instability maximum is inside the pupillary boundary B. In yet another embodiment, the controller C is adapted to calculate a total area of the respective pixels in the tear film stability map 300 having the stability index below a predefined area threshold, as well as a fraction of the total area to a total mapped ocular surface. Here the enabling condition is met when the fraction is greater than a predefined maximum.

In yet another embodiment, the controller C may be adapted to obtain a global stability value as a weighted sum of the stability index for respective pixels in the tear film stability map, such that weighting factors in the weighted sum are relatively higher in a central region (e.g. inside the pupillary boundary B) and relatively lower in a peripheral region of the ocular surface 12. Here the enabling condition is met when the global stability value is less than a predefined value.

Each of the threshold values (predefined pupil threshold, predefined area threshold, predefined maximum, predefined value etc.) may be selected through analysis of large sets of historical patient data. For example, this may be accomplished through a machine learning model 30, shown in FIG. 1. The controller C may interact with a cloud unit 32 and/or a remote server 34, for exchanging data across clinical sites. The cloud unit 32 may include one or more servers hosted on the Internet to store, manage, and process data. The remote server 34 may be a private or public source of information maintained by an organization, such as for example, a research institute, a company, a university and/or a hospital.

In summary, the system 10 provides an at-a-glance representation of ocular tear film stability through the tear film stability map 300, which is an integrated graphical display of two independent measurements - the tear film breakup time data and the blink completeness time series. The system 10 may be employed for screening of tear film stability assessment, dry eye disease screening, and as a screening tool to identify patients for whom advanced intraocular lens transplantation is indicated.

Referring to FIG. 1, the various components of the system 10 of FIG. 1 may communicate via a wireless network 36. The network 36 may be a bus implemented in various ways, such as for example, a serial communication bus in the form of a local area network. The local area network may include, but is not limited to, a Controller Area Network (CAN), a Controller Area Network with Flexible Data Rate (CAN-FD), Ethernet, blue tooth, WIFI and other forms of data connection. The network 36 may be a Wireless Local Area Network (LAN) which links multiple devices using a wireless distribution method, a Wireless Metropolitan Area Networks (MAN) which connects several wireless LANs or a Wireless Wide Area Network (WAN). Other types of connections may be employed.

The controller C of FIG. 1 includes a computer-readable medium (also referred to as a processor-readable medium), including a non-transitory (e.g., tangible) medium that participates in providing data (e.g., instructions) that may be read by a computer (e.g., by a processor of a computer). Such a medium may take many forms, including, but not limited to, non-volatile media and volatile media. Non-volatile media may include, for example, optical or magnetic disks and other persistent memory. Volatile media may include, for example, dynamic random-access memory (DRAM), which may constitute a main memory. Such instructions may be transmitted by one or more transmission media, including coaxial cables, copper wire and fiber optics, including the wires that comprise a system bus coupled to a processor of a computer. Some forms of computer-readable media include, for example, a floppy disk, a flexible disk, hard disk, magnetic tape, other magnetic medium, a CD-ROM, DVD, other optical medium, a physical medium, a RAM, a PROM, an EPROM, a FLASH-EEPROM, other memory chip or cartridge, or other medium from which a computer can read.

Look-up tables, databases, data repositories or other data stores described herein may include various kinds of mechanisms for storing, accessing, and retrieving various kinds of data, including a hierarchical database, a set of files in a file storage system, an application database in a proprietary format, a relational database management system (RDBMS), etc. Each such data store may be included within a computing device employing a computer operating system such as one of those mentioned above and may be accessed via a network in one or more of a variety of manners. A file system may be accessible from a computer operating system and may include files stored in various formats. An RDBMS may employ the Structured Query Language (SQL) in addition to a language for creating, storing, editing, and executing stored procedures, such as the PL/SQL language mentioned above.

The flowchart shown in the FIGS. illustrates an architecture, functionality, and operation of possible implementations of systems, methods, and computer program products according to various embodiments of the present disclosure. In this regard, each block in the flowchart or block diagrams may represent a module, segment, or portion of code, which comprises one or more executable instructions for implementing the specified logical function(s). It will also be noted that each block of the block diagrams and/or flowchart illustrations, and combinations of blocks in the block diagrams and/or flowchart illustrations, may be implemented by specific purpose hardware-based systems that perform the specified functions or acts, or combinations of specific purpose hardware and computer instructions. These computer program instructions may also be stored in a computer-readable medium that can direct a controller or other programmable data processing apparatus to function in a particular manner, such that the instructions stored in the computer-readable medium produce an article of manufacture including instructions to implement the function/act specified in the flowchart and/or block diagram blocks.

The numerical values of orders (e.g., of quantities or conditions) in this specification, including the appended claims, are to be understood as being modified in each respective instance by the term “about” whether or not “about” actually appears before the numerical value. “About” indicates that the stated numerical value allows some slight imprecision (with some approach to exactness in the value; about or reasonably close to the value; nearly). If the imprecision provided by “about” is not otherwise understood in the art with this ordinary meaning, then “about” as used herein indicates at least variations that may arise from ordinary methods of measuring and using such orders. In addition, disclosure of ranges includes disclosure of each value and further divided ranges within the entire range. Each value within a range and the endpoints of a range are hereby disclosed as separate embodiments.

The detailed description and the drawings or FIGS. are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims. Furthermore, the embodiments shown in the drawings, or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

What is claimed is:

1. A method of assessing tear film stability of an ocular surface with one or more imaging assemblies, and a controller with at least one processor and at least one non-transitory, tangible memory, the method comprising:
obtaining tear film breakup time data, via the one or more imaging assemblies;
converting the tear film breakup time data to a tear film breakup map represented as a polar data array such that respective elements in the polar data array represent a local tear film breakup time at a respective radial and angular location, via the controller;
obtaining a blink time series, via the one or more imaging assemblies;
converting the blink time series to an interblink interval map such that the respective elements in the interblink interval map represent an interblink time interval at different blink completeness levels, via the controller;
integrating the tear film breakup map with the interblink interval map to generate a tear film stability map, including calculating a stability index for respective pixels in the tear film stability map, via the controller; and
directing a remedial action when at least one enabling condition related to the tear film stability map is met.

2. The method of claim 1, further comprising, prior to integrating the tear film breakup map with the interblink interval map:
matching a respective scale of the interblink interval map and the tear film breakup map, the interblink interval map and the tear film breakup map having a respective corneal apex position, via the controller; and
aligning the interblink interval map and the tear film breakup map based on the respective corneal apex position, via the controller.

3. The method of claim 1, further comprising:
calculating the stability index as a ratio of the local tear film breakup time over the interblink time interval, via the controller.

4. The method of claim 1, further comprising:
calculating the stability index as a difference between the local tear film breakup time and the interblink time interval, via the controller.

5. The method of claim 1, further comprising:
setting the at least one enabling condition to be met when the stability index of at least one of the respective pixels inside a pupillary boundary in the tear film stability map is below a predefined pupil threshold.

6. The method of claim 1, further comprising:
identifying the respective pixels having a lowest value of the stability index as an instability maximum, via the controller; and
setting the at least one enabling condition to be met when the instability maximum is inside a pupillary boundary.

7. The method of claim 1, further comprising:
calculating a total area of the respective pixels in the tear film stability map having the stability index below a predefined area threshold, via the controller;
calculating a fraction of the total area to a total mapped ocular surface, via the controller; and
setting the at least one enabling condition to be met when the fraction is greater than a predefined maximum.

8. The method of claim 1, further comprising:
obtaining a global stability value as a weighted sum of the stability index for respective pixels in the tear film stability map such that weighting factors in the weighted sum are relatively higher in a central region and relatively lower in a peripheral region of the ocular surface, via the controller; and
setting the at least one enabling condition to be met when the global stability value is less than a predefined value.

9. The method of claim 1, further comprising:
selecting the remedial action to include a therapeutic treatment for dry eyes and/or a screening for advanced intraocular lens implantation.

10. The method of claim 1, further comprising:
selecting the one or more imaging assemblies to include a camera unit having a capturing frequency of at least 70 Hertz.

11. A system for assessing tear film stability of an ocular surface, the system comprising:
one or more imaging assemblies adapted to respectively obtain tear film breakup time data and a blink time series for the ocular surface;
a controller having a processor and tangible, non-transitory memory on which instructions are recorded, the controller being configured to:
convert the tear film breakup time data to a tear film breakup map represented as a polar data array such that respective elements in the polar data array represents a local tear film breakup time at a respective radial and angular location;
convert the blink time series to an interblink interval map such that the respective elements in the interblink interval map represent an interblink time interval at different blink completeness levels;
integrate the tear film breakup map with the interblink interval map to generate a tear film stability map, including calculating a stability index for respective pixels in the tear film stability map; and
direct a remedial action when at least one enabling condition related to the tear film stability map is met.

12. The system of claim 11, wherein the controller is adapted to, prior to integrating the tear film breakup map with the interblink interval map:
match a respective scale of the interblink interval map and the tear film breakup map, the interblink interval map and the tear film breakup map having a respective corneal apex position; and
align the interblink interval map and the tear film breakup map based on the respective corneal apex position.

13. The system of claim 11, wherein the controller is adapted to calculate the stability index as a ratio of the local tear film breakup time over the interblink time interval.

14. The system of claim 11, wherein the controller is adapted to calculate the stability index as a difference between the local tear film breakup time and the interblink time interval.

15. The system of claim 11, wherein the at least one enabling condition is met when the stability index of at least one of the respective pixels inside a pupillary boundary in the tear film stability map is below a predefined pupil threshold.

16. The system of claim 11, wherein the controller is adapted to identify the respective pixels having a lowest value of the stability index as an instability maximum, the at least one enabling condition being met when the instability maximum is inside a pupillary boundary.

17. The system of claim 11, wherein:
the controller is adapted to calculate a total area of the respective pixels in the tear film stability map having the stability index below a predefined area threshold;
the controller is adapted to calculate a fraction of the total area to a total mapped ocular surface; and
the at least one enabling condition is met when the fraction is greater than a predefined maximum.

18. The system of claim 11, wherein:
the controller is adapted to obtain a global stability value as a weighted sum of the stability index for respective pixels in the tear film stability map, such that weighting factors in the weighted sum are relatively higher in a central region and relatively lower in a peripheral region of the ocular surface; and
the at least one enabling condition is met when the global stability value is less than a predefined value.

19. The system of claim 11, wherein the remedial action includes a therapeutic treatment for dry eyes and/or a screening for advanced intraocular lens implantation.

20. A system for assessing tear film stability of an ocular surface, the system comprising:
one or more imaging assemblies adapted to respectively obtain tear film breakup time data and a blink time series for the ocular surface;
a controller having a processor and tangible, non-transitory memory on which instructions are recorded, the controller being configured to:
convert the tear film breakup time data to a tear film breakup map represented as a polar data array such that respective elements in the polar data array represents a local tear film breakup time at a respective radial and angular location;
convert the blink time series to an interblink interval map such that the respective elements in the interblink interval map represent an interblink time interval at different blink completeness levels;
match a respective scale of the interblink interval map and the tear film breakup map, the interblink interval map and the tear film breakup map having a respective corneal apex position;
align the interblink interval map and the tear film breakup map based on the respective corneal apex position;
integrate the tear film breakup map with the interblink interval map to generate a tear film stability map, including calculating a stability index for respective pixels in the tear film stability map; and
direct a remedial action when at least one enabling condition related to the tear film stability map is met, the remedial action including a therapeutic treatment for dry eyes and/or a screening for advanced intraocular lens implantation.
